(19) United States
(12) Patent Application Publication
(10) Pub. No.: US 2018/0104371 A1
(43) Pub. Date: Apr. 19, 2018

(54) METHOD FOR OPERATING A WICK-BASED VOLATILE COMPOSITION

(71) Applicant: The Procter & Gamble Company, Cincinnati, OH (US)

(72) Inventor: Erik John HASENOEHRL, Loveland, OH (US)

(21) Appl. No.: 15/292,499

(22) Filed: Oct. 13, 2016

(51) Int. Cl. A61L 9/03

(52) U.S. Cl. CPC A61L 9/037; A61L 2209/111; A61L 2209/135

(57) ABSTRACT

A method circulating a volatile composition throughout at least one room using an air handling device is provided. The volatile composition is delivered into the room from a volatile composition dispenser. The air handling device and the volatile composition dispenser are each communicably connectable with a central control unit. The volatile composition dispenser comprises a reservoir, a wick in fluid communication with the reservoir, and a heater disposed adjacent to the wick. The method includes a wick cleaning step where the heater is turned off for a duration of time of at least three hours.

### FIELD

The present disclosure is directed to a system and method for coupling the operations of a volatile composition dispenser and a smart appliance.

### BACKGROUND

Volatile composition dispensers exist in various forms, including non-energized devices that passively diffuse volatile compositions and energized devices that utilize energy in various forms to dispense, or assist in dispensing, a volatile composition. Some volatile composition dispensers include a wick that is used to transport a volatile composition from a reservoir and/or to evaporate the volatile composition therefrom. One issue with wick-based volatile composition dispensers is that it is often difficult to fill a room with a desired level of volatile composition, particularly over an extended period of time. Volatile composition dispensers have been modified in various ways in order to try and address this issue. However, it would be beneficial to provide a method that is able to improve the dispersion of a volatile composition dispensed from wick-based volatile composition dispensers.

### SUMMARY

### “Combinations”

A. A method of dispensing a volatile composition from a volatile composition dispenser, wherein the volatile composition dispenser is communicably connectable with a central control unit (CCU), the volatile composition dispenser comprising a reservoir, a wick in fluid communication with the reservoir, and a heater disposed adjacent to the wick, the method comprising the steps of:
(a) sending a first outgoing instruction from the central control unit to the volatile composition dispenser to turn on the heater of the volatile composition dispenser;
(b) sending a second outgoing instruction from the central control unit to the volatile composition dispenser to turn off the heater of the volatile composition dispenser in a wick cleaning mode for a duration of time of at least three hours between the hours of 10 p.m. and 4 a.m.; and
(c) sending a third outgoing instruction from the central control unit to the volatile composition dispenser to turn on the heater of the volatile composition dispenser after a first setpoint is reached; and
(d) optionally repeating steps (a) through (c).
B. The method of Paragraph A, wherein the volatile composition dispenser further comprises at least two reservoirs, each reservoir having a wick in fluid communication with the reservoir.
C. The method of Paragraph B, wherein the volatile composition dispenser further comprises a heater adjacent to each wick, wherein the second outgoing instruction includes turning each heater off during the same duration of time.
D. The method of any of Paragraphs A through C, wherein the volatile composition dispenser comprises an indicator, wherein the central control unit sends an instruction to the indicator to turn ON during the wick cleaning mode.
E. The method of Paragraph D, wherein the indicator is a light or sound associated with the volatile composition dispenser.
F. The method of Paragraph D, wherein the indicator is a notification on a user interface, wherein the user interface is selected from the group consisting of a phone, computer, tablet, or combination thereof.
G. The method of any of Paragraphs A through F, wherein the volatile composition dispenser includes an end-of-life sensor, wherein the method includes the steps of:

sending an end-of-life sensor level reading to the CCU; and

sending a notification to a user interface that alerts a user of the status of the cartridge or reminds the user to purchase a new cartridge.

H. The method of any of Paragraphs A through G, wherein the volatile composition dispenser includes an end-of-life sensor, wherein the method includes the steps of:

sending an end-of-life sensor level reading to the CCU; and

sending a third outgoing instruction to the CCU to order a replacement cartridge before the cartridge is depleted.

I. The method of any of Paragraphs A through H, wherein the central communication unit uses an algorithm to determine the optimal volatile composition emission and air handling device profile.
J. The method of any of Paragraphs A through I, wherein the volatile composition dispenser is communicably connectable with the central communication unit through a wireless communication link.
K. A method of dispensing a volatile composition from a volatile composition dispenser, wherein the volatile composition dispenser is communicably connectable with a central control unit (CCU), the volatile composition dispenser comprising a reservoir, a wick in fluid communication with the reservoir, and a heater disposed adjacent to the wick, the method comprising the steps of:
(a) using the CCU to learn when a user is away from the room where the volatile composition dispenser is disposed for at least three hours during the day;
(b) sending a first outgoing instruction from the central control unit to the volatile composition dispenser to turn on the heater of the volatile composition dispenser, wherein the central control unit is a learning central control unit;
(c) sending a second outgoing instruction from the central control unit to the volatile composition dispenser to turn off the heater of the volatile composition dispenser in a wick cleaning mode for a duration of time of at least three hours when the user is away from the room where the volatile composition dispenser is disposed for at least three hours; and
(d) sending a third outgoing instruction from the central control unit to the volatile composition dispenser to turn on the heater of the volatile composition dispenser after a first setpoint is reached;
(d) optionally repeating any of steps (a) through (d).
L. The method of Paragraph K, wherein the volatile composition dispenser comprises an indicator, wherein the central control unit sends an instruction to the indicator to turn ON during the wick cleaning mode, wherein the indicator is selected from the group consisting of a light, a sound, a notification to a user interface, and combinations thereof.
M. The method of Paragraph K or Paragraph M, wherein the volatile composition dispenser includes an end-of-life sensor, wherein the method includes the steps of:

sending an end-of-life sensor level reading to the CCU; and

sending a notification to a user interface that alerts a user of the status of the cartridge or reminds the user to purchase a new cartridge.

N. The method of any of Paragraphs K through M, wherein the volatile composition dispenser includes an end-of-life sensor, wherein the method includes the steps of:

sending an end-of-life sensor level reading to the CCU; and

sending a third outgoing instruction to the CCU to order a replacement cartridge before the cartridge is depleted.

O. A method of replacing a cartridge of a volatile composition dispenser, the volatile composition dispenser comprising a heater, a cartridge, and an end-of-life sensor, the cartridge comprising a reservoir for containing a volatile composition and a wick in fluid communication with the reservoir, wherein the volatile composition dispenser is communicably connectable with a central control unit (CCU), the method comprising the steps of:
(a) sending a first outgoing instruction from the central control unit to the volatile composition dispenser to turn on the heater of the volatile composition dispenser;
(b) sensing the level of volatile composition in the reservoir using an end-of-life sensor;
(c) sending the level details to the CCU;
(d) notifying a user through a user interface that the volatile composition level is low or needs to be replenished, wherein the user interface is a phone, tablet, computer, or combination thereof; and
(e) optionally repeating any of steps (a) through (d).
P. The method of Paragraph O further comprising the steps of:

sending a second outgoing instruction from the central control unit to the volatile composition dispenser to turn off the heater of the volatile composition dispenser after a second setpoint is reached; and

sending a third outgoing instruction from the central control unit to the volatile composition dispenser to turn on the heater of the volatile composition dispenser after a third setpoint is reached.

Q. The method of Paragraph O or P, further comprising the step of sending a fourth outgoing instruction to the CCU to order a replacement cartridge before the cartridge is depleted.
R. The method of any of Paragraphs O through Q, wherein the end-of-life sensor is selected from the group consisting of: a weight sensor, a visual sensor, a light sensor.
S. The method of any of Paragraphs O through R, wherein the end-of-life sensor is the heater, wherein the CCU records the ON time of the heater.
T. The method of any of Paragraphs O through S, wherein the central communication unit uses an algorithm to determine the optimal volatile composition emission and air handling device profile.

### BRIEF DESCRIPTION OF THE DRAWINGS

FIG. 1 illustrates exemplary components of a system, including the central communication unit and a volatile composition dispenser that communicates with the CCU through a wireless communication link.

FIG. 2 illustrates a volatile composition dispenser placed in a different room than a CCU.

FIG. 3 is a schematic front, elevation view of an exemplary volatile composition dispenser.

FIG. 4 is a schematic side, elevation view of an exemplary volatile composition dispenser.

FIG. 5 is a schematic top, plan view of an exemplary volatile composition dispenser.

FIG. 6 depicts multiple possible flows of signals from the components to the CCU.

FIG. 7 illustrates an exemplary CCU having the processor and the memory disposed within a housing 12.

FIG. 8 illustrates multiple exemplary flows of incoming signals from various components of the system to a remote memory.

### DETAILED DESCRIPTION

While the methods and systems of the present disclosure will be described more fully it is to be understood at the outset of the description which follows that persons of skill in the appropriate arts may modify the methods and systems herein described while still achieving the favorable results of described in the present disclosure. Accordingly, the description which follows is to be understood as being a broad, teaching disclosure directed to persons of skill in the appropriate arts, and not as limiting upon the present disclosure.

The methods include one or more volatile composition dispensers and a central communication unit (CCU) that is wirelessly connectable with the volatile composition dispenser(s). The volatile composition dispenser includes at least one cartridge having a reservoir for containing a volatile composition, a wick in fluid communication with the wick, and a seal engaged with the wick and the reservoir for preventing the volatile composition from escaping from the reservoir except through the wick. The volatile composition dispenser may also include a heater for assisting in the evaporation of the volatile composition from the wick. The volatile composition dispenser may include two or more cartridges for containing two or more different volatile compositions.

One shortcoming of wick-based volatile composition dispensers is that the wick can become clogged with heavier and lower volatility materials. The present disclosure includes a method of operating a volatile composition dispenser in a “wick cleaning mode” where the CCU turns off the heater or heaters of the volatile composition dispenser for a period of time to allow any volatile composition that is clogged in the wick to be released from the wick. When the heater is ultimately turned back on, the volatile composition refills the wick and is evaporated therefrom, resulting in increased noticeability or intensity of the volatile composition in the air. During the wick cleaning mode, it is preferable for the heaters to be turned off for at least two hours, more preferably at least three hours, most preferably at least four hours. It is preferable to activate the wick cleaning mode while the user is sleeping or away from the room or building where the volatile composition dispenser is located.

With a CCU that utilizes a machine learning algorithm and has learned when a user is not home or present in the room or building may be configured to turn OFF the heater(s) during that time instead of, or in addition to, turning the device off at night while the user is sleeping. By turning the volatile composition dispenser off at night or during a four hour period when the user is not home, the user does not notice that the volatile composition dispenser is turned off. Moreover, without wishing to be bound by theory, it is believed that by turning the volatile composition dispenser OFF while the user is sleeping, the user's scent receptors may be temporarily deactivated, which may improve the user's noticeability of the scent once the volatile composition dispenser is turned back ON.

During a “normal operation mode” of the volatile composition dispenser, the CCU may run various different programs. For example, the normal operation mode may include running the heater continuously to evaporate a volatile composition from the wick. In a volatile composition dispenser having at least two reservoirs and a wick in fluid communication with each reservoir, normal operation mode may include alternating operation from one heater to another for set periods of time. For example, a first heater may evaporate a volatile composition from a first wick for a set period of time, followed by turning the first heater off, then turning a second heater on to evaporate a volatile composition from a second wick for a set period of time. The volatile compositions may be different to prevent a user from becoming habituated to the volatile composition. This normal operation mode may be repeated and may alternate through all of the heaters in the volatile composition dispenser.

While the wick cleaning mode has been found to improve the noticeability or intensity of the volatile composition dispensed from the volatile composition, programming the volatile composition dispenser to turn ON and OFF can affect the life of the volatile composition. As a result of turning the wick-based volatile composition dispenser ON and OFF over time, the life of the volatile composition contained in the dispenser cannot be easily predicted. Further, a user who has become habituated may be comparatively insensitive to any reduction in volatiles concentrations that might occur approaching the end of life of the dispenser and corresponding depletion of the volatile compositions. Thus, the present disclosure includes a method of indicating to a user the remaining life of the volatile composition contained within the cartridge or indicating to a user that the volatile composition is near depletion. The indicator may serve as a reminder to the user to purchase a new cartridge or the indicator may send an instruction through the CCU to order the user a new cartridge that may be shipped to the user's home or office without the user having to take any active steps to purchasing a new cartridge. The indicator may be in the form of visual or audible indicator on the volatile composition dispenser, and/or an indicator on the user's phone or other user interface.

The end-of-life of the volatile composition may be measured in different ways using a sensor. For example, the end of life sensor may be selected from the group consisting of a weight sensor, visual sensor, light sensor, and the like. The end-of-life sensor may be the heater of the volatile composition dispenser and the central communication unit may record the ON time of the heater to calculate the amount of volatile composition dispensed from the volatile composition dispenser.

The methods of the present disclosure may also include pairing the operation of the volatile composition dispenser with the operation of a smart appliance or air handling device.

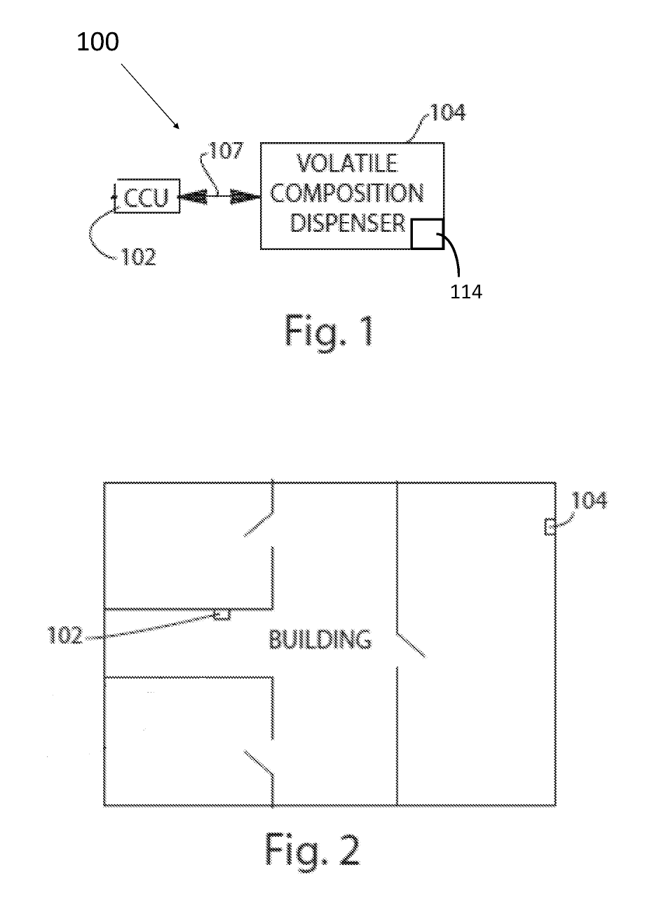

The systems and methods include a central communication unit (CCU) that is communicably connectable one or more volatile composition dispensers. FIG. 1 illustrates exemplary components of a system 100, including the central communication unit 102, a volatile composition dispenser 104 that communicates with the CCU 102 through a wireless communication link 107.

The CCU comprises a memory that is capable of storing set points and algorithms and a processor that is capable of running algorithms and accessing the stored set points from the memory. Various algorithms may be programmed depending upon the desired sequence of operations and the desired timing of each sequence.

The volatile composition dispenser 104 may be placed in any room of a building. With reference to FIG. 2, the volatile composition dispenser 104 may be placed in the same or different room from the CCU 102. The volatile composition dispenser 104 may be moved to different rooms at the user's convenience while remaining in communication with the CCU.

Volatile Composition Dispenser

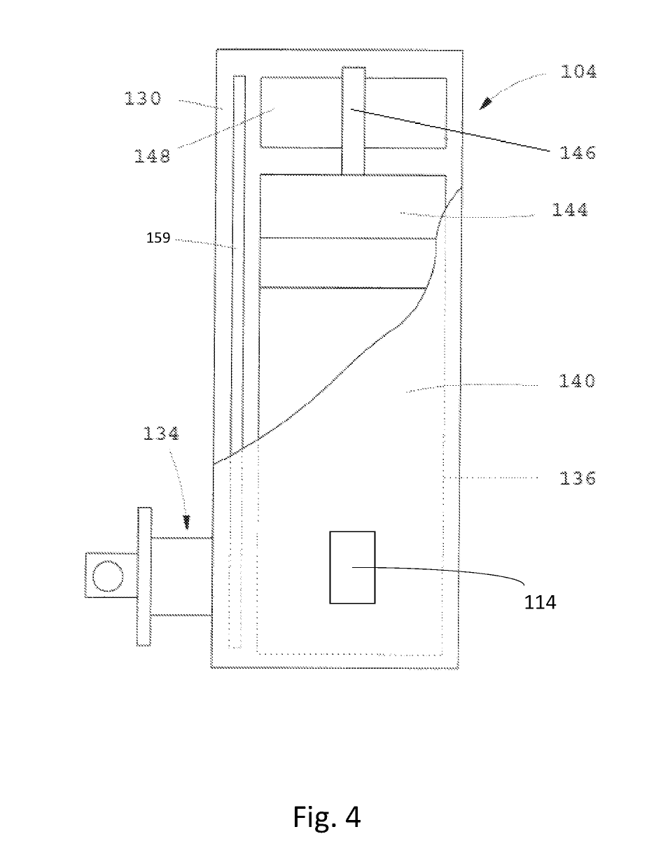

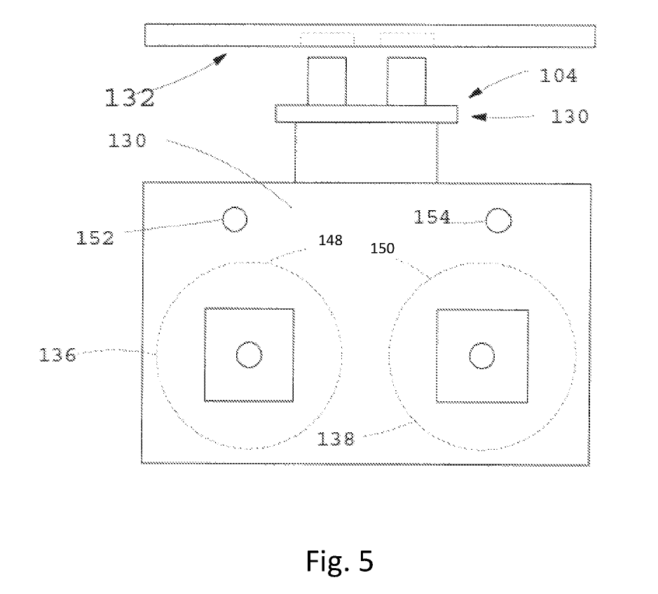

With reference to FIGS. 1 and 3-5, a volatile composition dispenser 104 may be used for the delivery of a volatile composition to the atmosphere or onto an inanimate surface.

Such volatile composition dispenser 104 may be configured in a variety of ways. The volatile composition dispenser may include a wireless communication module 114 in order to establish a wireless communication link 107 with various components of the system 100. The volatile composition dispenser may additionally include mesh network border router functionality.

One example of a volatile composition dispenser that can be used in accordance with the present disclosure is one that includes a wick. When used in such devices, the wick acts as a conduit to carry a volatile composition from a reservoir to a point of emission. A wick is generally porous, or includes pores, that provide for the flow of the volatile composition. Wicks can be made from a variety of materials, including but not limited to, cellulose fibers, metal, plastic, ceramic, graphite, and cloth. Synthetic materials, such as plastic, may be desirable because of their uniformity in performance Plastic materials that can be used to form porous wicks include, but are not limited to, high-density polyethylene (HDPE), polytetrafluoroethylene (PTFE), ultra-high molecular weight polyethylene (UHMW), nylon 6 (N6), polypropylene (PP), polyvinylidine fluoride (PVDF), and polyethersulfone (PES).

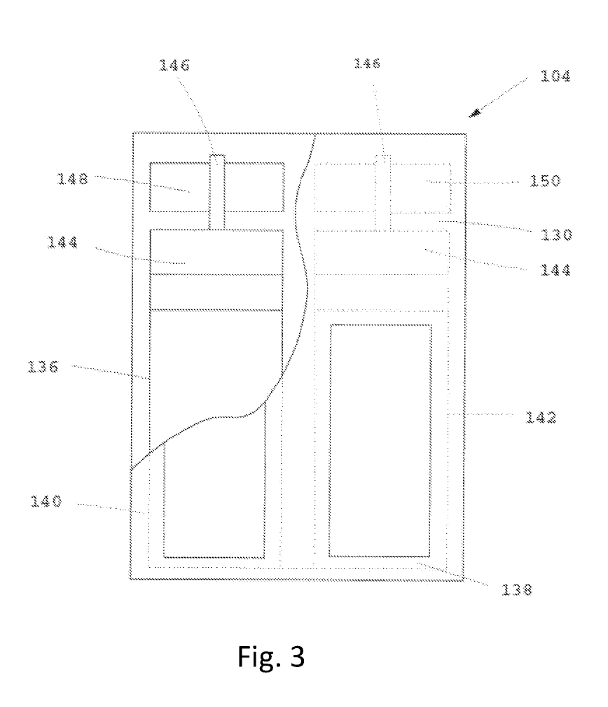

The volatile composition dispenser 104 comprises a housing 130, and the housing 130 is supported on an electrical outlet 132 by a plug 134 that is at least indirectly joined to the housing 130. The volatile composition dispenser 104 further comprises at least one container, or reservoir. For illustrative purposes only, FIGS. 3-5 show the volatile composition dispenser 104 having two reservoirs 136 and 138. The reservoirs 136 and 138 contain at least a first volatile composition 140 and a second volatile composition 142. The housing 130 may serve as a holder for the reservoirs 136 and 138 and any of the other components of the volatile composition dispenser described below.

The reservoirs 136 and 138 can comprise any suitable type of container, and can be made of any suitable material. Suitable materials for the reservoirs include, but are not limited to glass and plastic. The reservoirs 136 and 138 can comprise any type of container that is suitable for holding volatile compositions. The reservoirs 136 and 138 may be part of the housing 130, or they may be separate components that are removably joined to a portion of the volatile composition dispenser 104 such as the housing 130. It is also possible for a single reservoir to hold more than one type of volatile material. Such a reservoir could, for instance, have two or more compartments for volatile materials. In the embodiment shown in FIGS. 3-5, the reservoirs 136 and 138 comprise two separate bottles.

The reservoirs 136 and 138 in FIGS. 3-5 contain volatile compositions in the form of scented perfume oils. The reservoirs further comprise a seal 144 for containing the volatile material, and a wick 146 for dispensing the volatile material. The volatile composition dispenser 104 and/or the reservoirs 136 and 138 may further comprise an additional seal for covering the wick 146 of one or more of the volatile materials when the volatile material is not being emitted.

The wick and the reservoir may be configured as a cartridge that is releasably connectable with the housing. The cartridge can be replaced once the volatile composition in the cartridge is depleted.

In the case of scented materials or fragrances, the different scented materials can be similar, related, complementary, or contrasting. It may not be desirable, however, for the scented materials to be too similar if the different scented materials are being used in an attempt to avoid the problem of scent habituation; otherwise, the people experiencing the scents may not notice that a different scent is being emitted. The different scents can be related to each other by a common theme, or in some other manner For example, the different scents can all be floral, fruit scents, etc. An example of scents that are different, but complementary, might be a vanilla scent and a French vanilla scent.

With reference to FIGS. 3-5, the volatile composition dispenser further comprises a mechanism for activating the volatile materials from their “resting” state to an activated state. Such a component may include, but is not limited to, a component that volatilizes or heats the volatile materials. The volatile composition dispenser 104 may also contain a component, such as a fan, for diffusing or transporting the volatile materials into the environment or atmosphere. The volatile composition dispenser 104 may comprise a heater, a fan, or both, or some other type of mechanism.

The volatile composition dispenser 104 comprises at least one heating system or heater, such as heaters 148 and 150. The heaters 148 and 150 can comprise any suitable type of heater, and can be located in any suitable location in or relative to the volatile composition dispenser 104. The heaters 148 and 150 comprise heating elements that are in the form of circular rings that at least partially surround the wicks 146 protruding from the bottles of the volatile compositions.

The volatile composition dispenser 104 can comprise a switching mechanism 159 that changes the volatile material being emitted by the volatile composition dispenser 104. The switching mechanism 159 can comprise any suitable type of mechanism that causes the device to change the volatile material being emitted. In the embodiment shown, the switching mechanism controls the activation of the heaters so that the heater will be turned on for the volatile material that is desired to be emitted. Suitable switching mechanisms include, but are not limited to, analog timing circuitry, digital circuitry, combinations of analog and digital circuitry, microprocessors, and mechanical actuation switches such as shape memory alloys (NiTi wire) or bimetallic switches.

The volatile composition dispenser 104 can comprise a number of additional features. The device can be provided with indicators so that a person is further made aware that the volatile material being emitted has changed or has been depleted. Such indicators can be visual and/or audible. For example, in the case of scented materials, such an indicator may allow a person to see which scent is being emitted at a given time. For example, with reference to FIGS. 3-5, the indicators are in the form of lights 152 and 154. The indicator may be configured as a light, in-application notification, text message, voice message, voice or audible alert using, for example, an Alexa™ device.

The volatile composition dispenser 104 can be provided with additional user controls. The volatile composition dispenser 104 can include an “on/off” switch to allow a user to turn the device on and off without removing it from the electrical socket.

The volatile composition dispenser 104 may be movable to different rooms within a housing or building. Moreover, a house or building may include one or more volatile composition dispensers that are positioned in the same room or in different rooms.

Volatile Composition

The volatile composition may be an air freshening composition, including a perfume composition and/or a malodor control composition. The volatile composition may be an insect repellant.

The volatile composition may comprise volatile materials. Exemplary volatile materials include perfume materials, volatile dyes, materials that function as insecticides, essential oils or materials that acts to condition, modify, or otherwise modify the environment (e.g. to assist with sleep, wake, respiratory health, and like conditions), deodorants or malodor control compositions (e.g. odor neutralizing materials such as reactive aldehydes (as disclosed in U.S. 2005/0124512), odor blocking materials, odor masking materials, or sensory modifying materials such as ionones (also disclosed in U.S. 2005/0124512)).

The volatile composition may include perfume ingredients to provide a desirable scent in the air. The volatile composition includes a mixture of volatile aldehydes that are designed to deliver genuine malodor neutralization (and not function merely by covering up or masking odors). A genuine malodor neutralization provides a sensory and analytically measurable (e.g. gas chromatograph) malodor reduction. Thus, if the volatile composition delivers genuine malodor neutralization, the volatile composition will reduce malodors in the vapor and/or liquid phase. The volatile composition may comprise a mixture of volatile aldehydes that neutralize malodors in vapor and/or liquid phase via chemical reactions. Such volatile aldehydes are also called reactive aldehydes. Volatile aldehydes may react with amine-based odors, following the path of Schiff-base formation. Volatiles aldehydes may also react with sulfur-based odors, forming thiol acetals, hemi thiolacetals, and thiol esters in vapor and/or liquid phase.

The volatile composition may include various other ingredients, including, but not limited to: surfactants; acid catalysts; polymers; buffering agents; solubilizers; antimicrobial compounds; preservatives; wetting agents; aqueous carrier; diluents; the like; and combinations thereof.

The volatile composition may be configured to have a scent that matches a scent associated with a particular smart appliance. For example, the volatile composition may have a baby powder scent in an instance where the volatile composition dispenser is associated with a baby monitor or other device located within a baby's room. In another example, the volatile composition may have a laundry detergent or fabric softener scent in an instance where the volatile composition dispenser is associated with the operation of a washer or dryer.

Sensor

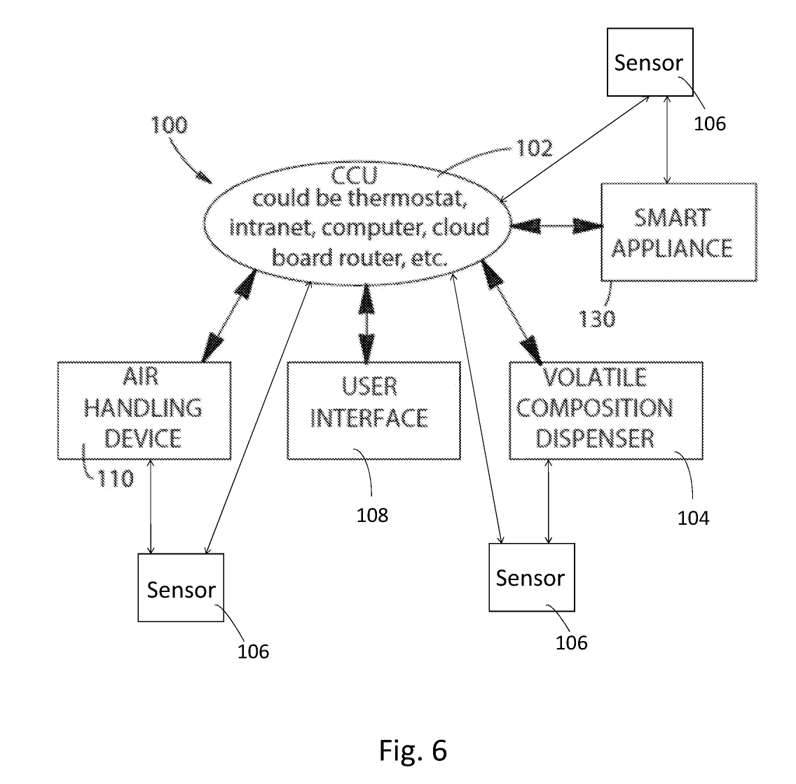

With reference to FIG. 6, one or more components of the system 100 may be associated with or may include a sensor 106. For example, the volatile composition dispenser 104, the CCU 102, an air handling device, or a smart appliance 130 may include one or more sensors 106. The sensors 106 may be configured to sense temperature, relative humidity, air quality, CO₂ levels, air particle counters, allergens and other air borne entities that have effect on human health, or the status of a component. The sensor 106 may include a wireless communication module in order to be communicably connectable with the CCU and various components of the system through a wireless communication link.

The sensor 106 may be powered by a power source. The sensor 106 may be powered independently from the volatile composition dispenser 104 or through the same power source of the volatile composition dispenser 104. The sensor 106 may be powered by a battery independently from the volatile composition dispenser 104 or by a battery configured to also power the volatile composition dispenser 104. The volatile composition dispenser and/or the sensor 106 may be powered through an electrical outlet. Battery power is used when the volatile composition dispenser is a mobile device that can be moved around from room to room or surface to surface. Moreover, a battery may be used to power the sensor 106 when the volatile composition dispenser is configured as a non-energized device that passively diffuses volatile compositions into the air.

The sensor is configured to send sensor measurements to the CCU in the form of incoming signals. The sensor measurements can be used in a variety of ways. For example, the sensor measurements may be viewed as live data; compared with set points, such as temperature set points in order to control the air handling device; or stored in a database for further analysis to recommend optimum set points taking comfort and energy efficiency into consideration.

A sensor may also be used to measure the end-of-life of the volatile composition contained within the cartridge. For example, the end of life sensor may be selected from the group consisting of a weight sensor, visual sensor, light sensor, and the like. The end-of-life sensor may be the heater of the volatile composition dispenser and the central communication unit may record the ON time of the heater to calculate the amount of volatile composition dispensed from the volatile composition dispenser.

FIG. 6 depicts multiple possible flows of signals from the components to the CCU. Sensor measurements can flow from a component through the CCU to a user interface for live local sensor measurements. Alternatively, the sensor measurements can also pass from the sensor through the CCU to a destination server on the internet where it is stored in memory or analyzed by a processor in order to send instructions to the various components, including, but not limited to, the air handling device 110 and volatile composition dispenser 104.

Central Communications Unit

The CCU 102 can be configured in various different ways. The CCU 102 may be configured to receive incoming signals from the one or more components of the system 100 and send outgoing instructions to one or more components of the system 100, for example the smart appliance(s) and/or the volatile composition dispenser(s).

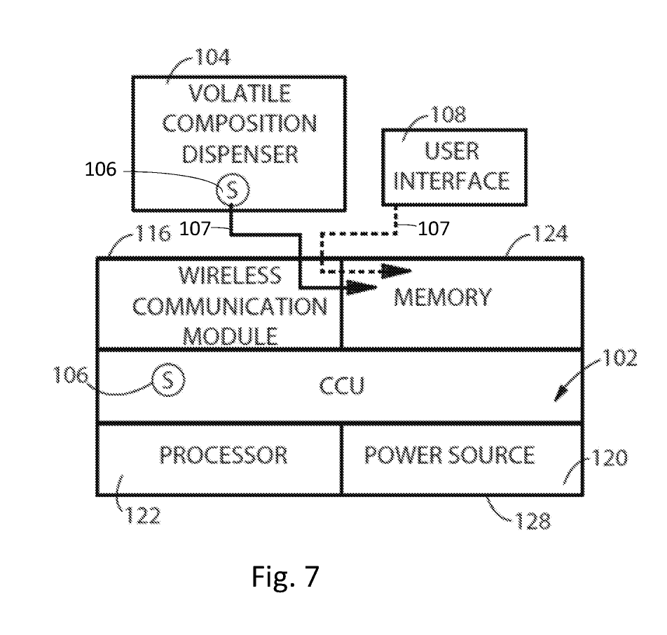

With reference to FIG. 7, the CCU 102 may be communicably connectable with various components of the system 100, including the sensor(s) 106, user interface(s) 108, the volatile composition dispenser 104, and/or smart appliances using a wireless communication link 107. Various wireless communication links may be used, including 802.11 (Wi-Fi), 802.15.4 (ZigBee, 6LoWPAN, Thread, JennetIP), Bluetooth, combinations thereof, and the like. Connection may be through an ad hoc Mesh Network protocol. The CCU 102 may include a wireless communication module 116 in order to establish a wireless communication link 107 with the CCU 102 with various components of the system. Any module known in the art for establishing the communication links can be utilized.

The CCU 102 may comprise a processor 122. The processor 122 may be configured and programmed to carry out and/or cause to be carried out the one or more advantageous functionalities of the system 100 described herein. The processor 122 may be physically disposed within a CCU 102 or may be remotely located on a computer, special computer, smart device such as a phone or tablet, server, intranet, border router, cloud-based system, the like, or combinations thereof. The processor 122 can carry out algorithms stored in local memory; special-purpose processors or application-specific integrated circuits; algorithms carried out or governed remotely by central servers, or cloud-based systems, such as by virtue of running a Java virtual machine that executes instructions provided from a cloud server using Asynchronous JavaScript and XML or similar protocols.

The CCU 102 may comprise a memory 124. The memory may be configured to store set points; incoming signals, such as sensor measurements and status indicators; algorithms;

and the like. The memory may be a local memory within the CCU 102 such as a flash drive, hard drive, read only memory, or random access memory. Or, the memory may be configured as remote memory on a computer, smart device such as a phone or tablet, on a server, or on cloud-based system. The memory 124 can be accessible to the processor 122 in a variety of ways.

The processor and/or the memory of the CCU 102 may be disposed within a housing of the CCU 102. The CCU 102 may be connected with or separate from various components of the system 100. For example, the CCU 102 may be physically connected with the smart appliance 130 or the volatile composition dispenser 104. The CCU 102 may be permanently positioned in a building in a separate room or location from other components such as the smart appliance 130, the volatile composition dispenser 104, and/or an air handling device, for example.

The CCU may include a clock or may be communicably connectable with a clock on a computer, smart device, or on the internet.

FIG. 7 illustrates an exemplary CCU 102 having the processor 122 and the memory 124 disposed within a housing 128. The CCU 102 shown in FIG. 85 may be disposed on or within a volatile composition dispenser 104, an air handling device 110, and/or a smart appliance. While FIG. 7 illustrates a processor 122 and a memory 124 disposed within the housing 128, it is to be appreciated that the processor 122 and/or the memory 124 may be remotely located relative to the CCU 102.

Incoming signals may pass through a CCU unit comprising a transmitter that transmits the incoming signals to the remote memory. Incoming signals may also be directly received by a component that is wirelessly communicating with the component sending the signals.

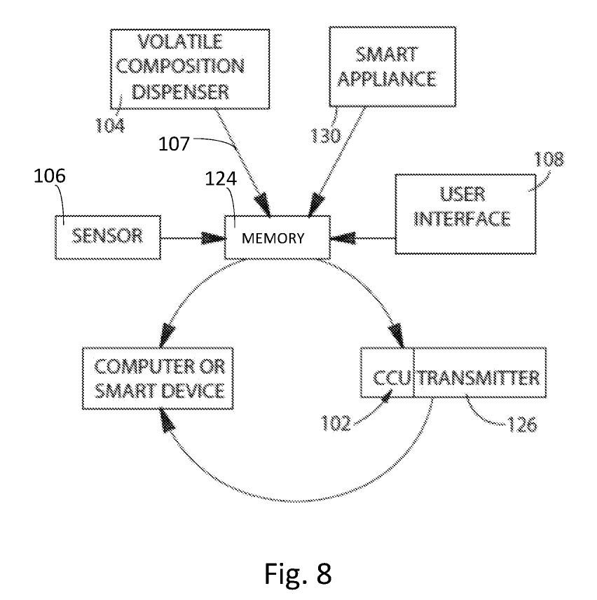

FIG. 8 illustrates multiple exemplary flows of incoming signals from various components of the system 100 to a remote memory. The incoming signals may flow directly from the smart appliance 130, volatile composition dispenser 104, or various other components to a computer or smart device through a wireless communication link or through a transmitter of the CCU to a remote memory. The processor of the CCU may access the incoming signals from the memory 124. The processor may access the memory 124 through a wired or wireless communication link.

The processor 122 may be configured to compare incoming signals to set points stored in the memory 124. The processor is able to retrieve stored set points from the memory 124 to compare.

Wick-based volatile composition dispensers, such as the FEBREZE® NOTICEABLES® air freshener, may suffer from decreased scent intensity and noticeability over time as a result of the wick clogging with portions of the volatile composition that have a lower volatility. It has been found that using an algorithm that includes turning the one or more heaters of the volatile composition dispenser off for a duration of time allows at least a portion of the volatile composition in the wick of the air freshener to release from the wick back into the reservoir, unclogging the wick, herein referred to as “wick cleaning mode”. As a result, when the heater is turned back on, the scent intensity and noticeability improve. More preferably, an algorithm that includes turning the one or more heaters of the volatile composition dispenser off for at least three hours or more preferably at least four hours can improve the scent intensity or noticeability from the volatile composition dispenser. Most preferably, it has been found that turning the one or more heaters of the volatile composition dispenser off for at least three hours or more preferably at least four hours while a user is sleeping, such as during the hours of 10 p.m. and 4 a.m., can improve the scent intensity or noticeability from the volatile composition dispenser. If the volatile composition dispenser includes more than one heater, such as when more than one wick and reservoir are present, all of the heaters may be turned off during the “wick cleaning mode”.

During wick cleaning mode, an indicator on the volatile composition dispenser may be turned ON to alert the user that the volatile composition dispenser is in “wick cleaning mode”.

A non-limiting example may include using a volatile composition dispenser, such as a FEBREZE® NOTICEABLES® plug-in air freshener for exemplary purposes only, comprising one or more of a 802.15.4 or 802.15.11 wireless radio with an algorithm that is configured to emit a volatile composition for a duration of time. After the time set point is reached, the algorithm causes the FEBREZE® NOTICEABLES® plug-in air freshener to stop emitting the volatile composition. The FEBREZE® NOTICEABLES® plug-in air freshener may emit the volatile composition by turning on one or more heaters of the air freshener. To stop emitting the volatile composition, the one or more heaters of the air freshener may be turned off. The cycle may be repeated.

An exemplary algorithm may include sending a first outgoing instruction from the central control unit to the volatile composition dispenser to turn on the heater of the volatile composition dispenser; sending a second outgoing instruction from the central control unit to the volatile composition dispenser to turn off the heater of the volatile composition dispenser in a wick cleaning mode for a duration of time of at least three hours between the hours of 10 p.m. and 4 a.m.; and sending a third outgoing instruction from the central control unit to the volatile composition dispenser to turn on the heater of the volatile composition dispenser after a first setpoint is reached.

The CCU may be a learning CCU that learns when a user is away from the room where the volatile composition dispenser is disposed for at least three hours during the day. With a learning CCU, the outgoing instruction from the central control unit that turns off the heater of the volatile composition dispenser may occur during the at least three hour period when the user is away from the room where the volatile composition dispenser is disposed for at least three hours.

The volatile composition dispenser comprises an indicator, wherein the central control unit sends an instruction to the indicator to turn ON during the wick cleaning mode. The indicator may be a light or sound associated with the volatile composition dispenser. The indicator may be a notification on a user interface.

As will be discussed in more detail below, a sensor or sensors may be used to predict or determine the end of life of the volatile composition in the cartridge. For example, the end of life of the volatile composition may be measured by a liquid level sensor, a weight sensor, visual or light sensor, and the like.

An exemplary algorithm may include the use of an end-of-life sensor to alert the user when the cartridge needs to be replaced or to automatically order a new cartridge before the current cartridge is depleted. The algorithm may include sending an end-of-life sensor level reading to the CCU and sending a notification that alerts a user of the status of the cartridge life or reminds the user to purchase a new cartridge. The algorithm may also include sending a fourth outgoing instruction to the CCU to order a replacement cartridge before the cartridge is depleted. This algorithm may be used in combination with any other algorithm described herein.

The central control unit may thereby send an instruction to an indicator on the volatile composition dispenser to turn ON to alert the user the cartridge needs to be replaced. The indicator may be a light or sound associated with the volatile composition dispenser. The indicator may also be a notification on a user interface.

Set points may include a duration of time; a level of volatile composition dispensed; a current condition or status of a component, or the like. The processor may utilize various components in order to determine when a set point has been reached. For example, the processor may utilize a clock, which may be connected with the CCU or remotely located on a computer, smart device, or on the internet and may be used to measure a duration of time. In another example, a sensor may be used to measure various conditions, such as the current temperature or level of volatile composition in the air. The sensor may be located on any component of the system or remote from the components of the system. The set point may also be based on a condition of a component. For example, a set point may be that a component is turned ON or OFF, which may signal the CCU to move on to the next step in the algorithm. The status of a component may be communicated to the CCU.

The memory 124 may be configured to store multiple set points. For example, there may be different set points for different times, time periods of a day and there may be different set points for different days of the week. The processor may include a clock in determine which set point is to be used for a particular time of day and/or day of the week.

The processor may be configured to use outgoing signals or sensor measurements from different components located within a house or building at different times of the day and/or different days of the week. For example, a first volatile composition dispenser may be located in a first room of a house and may be coupled with a first smart appliance. In addition, a second volatile composition dispenser may be located in a second room of the house or in the same room as the first room and may be in communication with the CCU. The CCU may be programmed concurrently control the first and second volatile composition dispensers.

The CCU may be configured as a thermostat such as the CCU shown in FIG. 2. Power for the thermostat may be scavenged from the control wires that connect the air handling device to the thermostat. The thermostat may include a processor or memory, or the thermostat may communicate with a remote processor and/or memory. The thermostat may include a user interface. The thermostat may be a NEST® learning thermostat, a LUTRON® thermostat etc.

The processor may compute optimal set points from an algorithm based on user preferences of volatile composition levels based on historical sensor measurements, historical set points, and known information on energy efficiency, comfort, and volatile composition levels. A machine learning algorithm can learn a user(s) preferred set points at various times of day and/or days of the week and/or can be used to program a more energy efficient algorithm, for example. An exemplary learning system is used in a NEST® learning thermostat. An exemplary learning system is also described in U.S. Pat. No. 9,115,908. The processor then transmits the optimal set points to the memory which then stores the set points for control of the HVAC.

Devices, including the air handling device, volatile composition dispenser, and/or smart appliance(s), of the system may interact with each other through the CCU such that events detected by one device may influence actions of another device or the current status of one device may influence actions of another device.

User Interface

The systems and methods of the present disclosure may include one or more user interfaces 108. The user interface 108 may be configured in various different forms. A user can interact with the user interface 108 to adjust set points as well as connect the sensors 106 through the CCU 102 for viewing of live sensor data on the user interface. The CCU 102 could also connect to the internet or intranet and pass through information, such as sensor measurements and the set points to a server for the purpose of remote monitoring on a user interface 108.

Where the CCU is configured as a thermostat, the thermostat may include a user interface where the user can adjust temperature set points by pushing buttons or turning dials, for example.

The user interface may be configured as a program, HTML website, or a Native application that is accessible by a user through a computer or handheld smart device. A handheld smart device may include an iPhone®, iPad®, or an Android® or Microsoft® based system. The user interface may be accessible on a computer such as a desktop, laptop, or tablet.

The system of the present disclosure may include a handheld smart device or computer that comprises the CCU 102, including the processor 122, memory 124, and/or user interface 108.

Smart Appliances

Various different smart appliances may also be connected with the system and included in the methods of the present disclosure. The smart appliances 130 may be communicably connectable with the CCU. Smart appliances may include refrigerators, washers, dryers, dishwashers, microwaves, stoves, ovens, garbage disposal, stereos, televisions, cable or satellite boxes, baby monitors, vacuum cleaners, security systems, lights, garage door openers, doorbell, indoor or outdoor sprinklers or irrigation systems, and the like.

The CCU may also be configured to control the volatile composition dispenser based upon the current status of a smart appliance. The smart appliances may be configured to send an alert in the form of an incoming signal to the CCU, alerting the CCU that a particular smart appliance has been turned on, turned off, or that a cycle of a smart appliance has started or ended. The smart appliance may also include sensors that are configured to send incoming signals to the CCU that alert the CCU as to current status of the smart appliance.

The CCU may also be configured initiate an algorithm that may include controlling a smart appliance based on sensor measurements sent to the CCU from other components of the system.

Air Handling System

The system 100 may include an air handling device. Air handling device includes a fan. The air handling device may provide heating, ventilation, air condition (HVAC) and/or air handling to an enclosure, such as a single-family home, apartment, office building, business, and the like. The air handling device 110 may be configured as a forced air type heating and cooling system. However, the air handling device may be configured in various different ways. For example, the air handling device may be configured in the form of a radiant heat based system; heat-pump based system; fan, including ceiling fan or portable fan; portable air conditioner; and/or portable heater.

The air handling device may have one or more controls. The controls on the air handling device may include turn ON/OFF the heater, turn ON/OFF the air conditioner, and/or turn ON/OFF the fan only. It is to be appreciated that the fan may also run when the heater or air conditioner is ON in order to push the heated or cooled air throughout the at least one room of the building.

The air handling device may include a wireless communication module in order to be wirelessly connected with various components of the system, such as the CCU, the volatile composition dispenser 104, and/or the smart appliance, through a wireless communication link.

Exemplary systems and methods that include coupling the operation of an air handling device with the operation of a volatile composition dispenser are described in U.S. Patent Application, entitled “SYSTEMS AND METHODS FOR COUPLING THE OPERATIONS OF AN AIR HANDLING DEVICE AND A VOLATILE COMPOSITION DISPENSER”, Attorney Docket No. 14052, filed on Oct. 8, 2015. Exemplary systems and methods for controlling an air handling device with a volatile composition dispenser are described in U.S. Patent Application, entitled “VOLATILE COMPOSITION DISPENSER HAVING A TEMPERATURE SENSOR TO REMOTELY CONTROL AN AIR HANDLING DEVICE”, Attorney Docket No. 14051, filed on Oct. 8, 2015.

Values disclosed herein as ends of ranges are not to be understood as being strictly limited to the exact numerical values recited. Instead, unless otherwise specified, each numerical range is intended to mean both the recited values, any integers within the specified range, and any ranges with the specified range. For example a range disclosed as “1 to 10” is intended to mean “1, 2, 3, 4, 5, 6, 7, 8, 9, and 10.” It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Values disclosed herein as ends of ranges are not to be understood as being strictly limited to the exact numerical values recited. Instead, unless otherwise specified, each numerical range is intended to mean both the recited values, any integers within the specified range, and any ranges with the specified range. For example a range disclosed as “1 to 10” is intended to mean “1, 2, 3, 4, 5, 6, 7, 8, 9, and 10.”

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of dispensing a volatile composition from a volatile composition dispenser, wherein the volatile composition dispenser is communicably connectable with a central control unit (CCU), the volatile composition dispenser comprising a reservoir, a wick in fluid communication with the reservoir, and a heater disposed adjacent to the wick, the method comprising the steps of:
(a) sending a first outgoing instruction from the central control unit to the volatile composition dispenser to turn on the heater of the volatile composition dispenser;
(b) sending a second outgoing instruction from the central control unit to the volatile composition dispenser to turn off the heater of the volatile composition dispenser in a wick cleaning mode for a duration of time of at least three hours between the hours of 10 p.m. and 4 a.m.; and
(c) sending a third outgoing instruction from the central control unit to the volatile composition dispenser to turn on the heater of the volatile composition dispenser after a first setpoint is reached; and
(d) optionally repeating steps (a) through (c).

2. The method of claim 1, wherein the volatile composition dispenser further comprises at least two reservoirs, each reservoir having a wick in fluid communication with the reservoir.

3. The method of claim 2, wherein the volatile composition dispenser further comprises a heater adjacent to each wick, wherein the second outgoing instruction includes turning each heater off during the same duration of time.

4. The method of claim 1, wherein the volatile composition dispenser comprises an indicator, wherein the central control unit sends an instruction to the indicator to turn ON during the wick cleaning mode.

5. The method of claim 4, wherein the indicator is a light or sound associated with the volatile composition dispenser.

6. The method of claim 4, wherein the indicator is a notification on a user interface, wherein the user interface is selected from the group consisting of a phone, computer, tablet, or combination thereof.

7. The method of claim 1, wherein the volatile composition dispenser includes an end-of-life sensor, wherein the method includes the steps of:
sending an end-of-life sensor level reading to the CCU; and
sending a notification to a user interface that alerts a user of the status of the cartridge or reminds the user to purchase a new cartridge.

8. The method of claim 1, wherein the volatile composition dispenser includes an end-of-life sensor, wherein the method includes the steps of:
sending an end-of-life sensor level reading to the CCU; and
sending a third outgoing instruction to the CCU to order a replacement cartridge before the cartridge is depleted.

9. The method of claim 1, wherein the central communication unit uses an algorithm to determine the optimal volatile composition emission and air handling device profile.

10. The method of claim 1, wherein the volatile composition dispenser is communicably connectable with the central communication unit through a wireless communication link.

11. A method of dispensing a volatile composition from a volatile composition dispenser, wherein the volatile composition dispenser is communicably connectable with a central control unit (CCU), the volatile composition dispenser comprising a reservoir, a wick in fluid communication with the reservoir, and a heater disposed adjacent to the wick, the method comprising the steps of:
(a) using the CCU to learn when a user is away from the room where the volatile composition dispenser is disposed for at least three hours during the day;
(b) sending a first outgoing instruction from the central control unit to the volatile composition dispenser to turn on the heater of the volatile composition dispenser, wherein the central control unit is a learning central control unit;
(c) sending a second outgoing instruction from the central control unit to the volatile composition dispenser to turn off the heater of the volatile composition dispenser in a wick cleaning mode for a duration of time of at least three hours when the user is away from the room where the volatile composition dispenser is disposed for at least three hours; and
(d) sending a third outgoing instruction from the central control unit to the volatile composition dispenser to turn on the heater of the volatile composition dispenser after a first setpoint is reached;
(d) optionally repeating any of steps (a) through (d).

12. The method of claim 11, wherein the volatile composition dispenser comprises an indicator, wherein the central control unit sends an instruction to the indicator to turn ON during the wick cleaning mode, wherein the indicator is selected from the group consisting of a light, a sound, a notification to a user interface, and combinations thereof.

13. The method of claim 11, wherein the volatile composition dispenser includes an end-of-life sensor, wherein the method includes the steps of:
sending an end-of-life sensor level reading to the CCU; and
sending a notification to a user interface that alerts a user of the status of the cartridge or reminds the user to purchase a new cartridge.

14. The method of claim 11, wherein the volatile composition dispenser includes an end-of-life sensor, wherein the method includes the steps of:
sending an end-of-life sensor level reading to the CCU; and
sending a third outgoing instruction to the CCU to order a replacement cartridge before the cartridge is depleted.

15. A method of replacing a cartridge of a volatile composition dispenser, the volatile composition dispenser comprising a heater, a cartridge, and an end-of-life sensor, the cartridge comprising a reservoir for containing a volatile composition and a wick in fluid communication with the reservoir, wherein the volatile composition dispenser is communicably connectable with a central control unit (CCU), the method comprising the steps of:
(a) sending a first outgoing instruction from the central control unit to the volatile composition dispenser to turn on the heater of the volatile composition dispenser;
(b) sensing the level of volatile composition in the reservoir using an end-of-life sensor;
(c) sending the level details to the CCU;
(d) notifying a user through a user interface that the volatile composition level is low or needs to be replenished, wherein the user interface is a phone, tablet, computer, or combination thereof; and
(e) optionally repeating any of steps (a) through (d).

16. The method of claim 15 further comprising the steps of:
sending a second outgoing instruction from the central control unit to the volatile composition dispenser to turn off the heater of the volatile composition dispenser after a second setpoint is reached; and
sending a third outgoing instruction from the central control unit to the volatile composition dispenser to turn on the heater of the volatile composition dispenser after a third setpoint is reached.

17. The method of claim 15, further comprising the step of sending a fourth outgoing instruction to the CCU to order a replacement cartridge before the cartridge is depleted.

18. The method of claim 15, wherein the end-of-life sensor is selected from the group consisting of: a weight sensor, a visual sensor, a light sensor.

19. The method of claim 15, wherein the end-of-life sensor is the heater, wherein the CCU records the ON time of the heater.

20. The method of claim 15, wherein the central communication unit uses an algorithm to determine the optimal volatile composition emission and air handling device profile.
